(19) United States
(12) Patent Application Publication
(10) Pub. No.: US 2009/0234273 A1
(43) Pub. Date: Sep. 17, 2009

(54) Surgical trocar with feedback

(76) Inventors: Alfred Intoccia, Nashua, NH (US); Isaac Ostrovsky, Wellesley, MA (US); Victor Shukhat, Canton, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/382,061

(22) Filed: Mar. 6, 2009

Related U.S. Application Data
(60) Provisional application No. 61/064,625, filed on Mar. 17, 2008.

(51) Int. Cl. A61B 17/34

(52) U.S. Cl. 604/22

(57) ABSTRACT

A medical device is disclosed. The medical device includes a handle configured to be grasped by a human hand, and an elongate obturator coupled to the handle. The obturator includes a proximal end proximate the handle and a distal end extending away from the handle. The distal end of the obturator includes a tip configured to pierce a body. The medical device also includes a vibration generating device coupled to the obturator. The vibration generating device is configured to induce a vibration having a frequency and an amplitude on the obturator.

### CROSS REFERENCE TO RELATED APPLICATIONS

This application is based upon and claims the benefit of priority under 35 U.S.C. §119(e) of U.S. Provisional Application No. 61/064,625 to Intoccia et al. filed on Mar. 17, 2008.

### FIELD OF THE INVENTION

This disclosure relates generally to a medical device, such as a trocar and, more particularly, to a surgical trocar having feedback capability, and methods of using the same.

### BACKGROUND OF THE INVENTION

In recent years, minimally invasive surgical techniques (e.g., laparoscopic surgery) have become a standard method for performing several common surgical procedures. These procedures are generally performed by using a puncture on the body surface to deliver desired surgical tools to a worksite inside the body. For instance, for removal of the gall bladder, laparoscopic cholecystectomy may be performed in place of conventional surgical cholecystectomy. In place of an abdominal incision to perform conventional cholecystectomy, laparoscopic cholecystectomy is performed using a laparoscope inserted into the abdomen through a puncture created on the abdomen. As compared to conventional open surgical procedures, patient discomfort and recovery time in laparoscopic procedures are generally lower due to the absence of a large incision. Although patient discomfort and associated trauma may have decreased due to laparoscopic procedures, the potential for serious complications (or injuries) still exists during laparoscopic procedures.

Laparoscopic surgery may begin by placing a specialized needle, for example a veress needle, into the abdominal cavity and filling the abdominal cavity with carbon dioxide gas in a process called insufflation. Insufflation elevates and holds the abdominal wall away from internal structures. Insertion of a trocar through the abdominal wall may follow. The trocar is a needle like device with a sharp tip protruding from a tubular casing (canulla). The sharp tip of the trocar is used to puncture the abdominal wall. After creating the puncture, the sharp needle may be retracted leaving the canulla in the puncture to provide an access port into the abdominal cavity. This process is sometimes referred to as a primary trocar creating a primary puncture. The laparoscope, equipped with a camera, may be inserted into the abdominal cavity through the canulla port of the primary trocar. The laparoscope may include a metal tube equipped with a camera and one or more access ports that allow a myriad of surgical instruments to be delivered into the abdominal cavity. The camera provides visualization for the surgeon during the laparoscopic procedure. The surgical instruments delivered through the laparoscope may be used to dissect, tie, clip, cauterize blood vessels, or perform any other desired procedure in the abdominal cavity during the procedure. A second and third trocar (secondary trocars) may also be similarly inserted into the abdominal wall. These insertions may be guided by the camera of the laparoscope inserted through the primary puncture. The secondary cannula ports may be used for retractors and graspers which retract tissue, for irrigation-suction devices, or for other desired tools or devices.

The primary trocar is typically inserted into the body using a blind puncture. Studies indicate that some complications that arise in laparoscopic surgery arise during blind insertion of the primary trocar (see “Laparoscopic Trocar Injuries: A report from a U.S. Food and Drug Administration (FDA) Center for Devices and Radiological Health (CDRH) Systematic Technology Assessment of Medical Products (STAMP) Committee,” by Janie Fuller et al., report available at http://www.fda.gov/cdrh/medicaldevicesafety/stamp/trocar.html). The blind insertion of the primary trocar may use a technique referred to as a controlled jab. The force required for the controlled jab can vary from patient to patient and from trocar to trocar depending, among others, upon the sharpness of the trocar blade. Due to the differences in density between different types of body tissue, the required force may also vary as the tip penetrates through different layers (for example, skin-fat-muscle-peritoneal layers) of the abdominal wall. For successful blind insertion, the surgeon must apply sufficient force under adequate control to stop the trocar movement upon penetration. When the trocar passes from one layer of tissue to another, there may be an abrupt change in piercing resistance. A surgeon may be unaware of, or may be unable to respond quickly to this changing piercing resistance, with the result that the sharp trocar tip may be thrust into the abdominal cavity, potentially causing injury to the organs or vessels contained therein. There may be numerous other medical procedures where a blind puncture may be used. For example, a blind puncture by a trocar may be used in treatment of female urinary incontinence, Tension free Vaginal Tape (TVT) procedure, or Transobturator Tape (TOT).

There are a number of improvements that have been recommended to reduce the likelihood of injury due to blind insertion of a trocar. While some of these techniques have focused on a modified surgical technique to insert the trocar into the abdominal cavity, most improvements focus on modified trocar designs. Some of these changes include modifications on the shape and sharpness of the sharp tip to reduce the amount of force needed to pierce the body cavity wall, and providing a sheath for the sharp tip to cover the tip after piercing the wall. Although these modified tip designs may reduce the likelihood of injury due to blind trocar insertion, the potential for these injuries still exists (see id.).

The present disclosure provides surgical trocars having feedback capability, and methods of using the same to avoid some or all of the aforementioned shortcomings of existing devices.

### SUMMARY OF THE INVENTION

In an exemplary embodiment of the present disclosure, a medical device is disclosed. The medical device includes a handle configured to be grasped by a human hand, and an elongate obturator coupled to the handle. The obturator includes a proximal end proximate the handle and a distal end extending away from the handle. The distal end of the obturator includes a tip configured to pierce a body. The medical device also includes a vibration generating device coupled to the obturator. The vibration generating device is configured to induce a vibration having a frequency and an amplitude on the obturator.

Various embodiments of the medical device may include one or more of the following aspects: the vibration generating device is embedded in the medical device; the obturator is configured to change the amplitude of vibration of the obturator as the obturator pierces the body; the medical device is further configured to transmit the vibration to the handle; the obturator is configured to vary the amplitude of vibration as a function of a density of a layer of tissue that the obturator pierces through; the proximal end of the obturator extends into the handle; the vibration generating device is physically coupled to the obturator; the vibration generating device includes at least one of an electric motor and a solenoid; the medical device includes a sensor coupled to the obturator, wherein the sensor is configured to detect a vibration of the obturator; the sensor includes one of a pressure sensor and an accelerometer; the sensor transmits signals indicative of the detected vibrations to a signal processing device; the signal processing device is embedded in the medical device; the signal processing device is embedded in the handle; the signal processing device is a standalone device separate from the medical device; the sensor wirelessly transmits the signals to the signal processing device; the signal processing device is configured to indicate information related to the position of the obturator within the body based on the signal; the information includes the type of tissue that the obturator pierces through; the information is indicated using a visual or an audio signal; the signal processing device is configured to indicate the proximity of the obturator to a bone in the body as the obturator pierces the body; the signal processing device is configured to indicate a contact of the obturator with a bone in the body; and the medical device further includes a vibration isolation bushing between the obturator and the handle.

In another exemplary embodiment of the present disclosure, a method of using a medical device to create a puncture in a body is disclosed. The method includes grasping a handle of the medical device with a hand, the handle being coupled to an elongate obturator. The method also includes activating a vibration generating device coupled to the obturator. The vibration generating device induces a vibration having a frequency and an amplitude on the obturator. The method further includes piercing the body with the obturator, detecting a vibration of the obturator during the piercing, and controlling the piercing of the body based on the detected vibration of the obturator.

Various embodiments of the method of using the medical device may include one or more of the following aspects: controlling the piercing includes detecting a variation of the amplitude of the vibration as the obturator pierces into the body; controlling the piercing includes detecting a frequency of vibration induced in the obturator; controlling the piercing includes identifying the type of tissue the obturator is piercing through based on the vibration of the obturator; controlling the piercing includes detecting contact of the obturator with a bone in the body based on the vibration of the obturator; detecting the vibration of the obturator includes detecting the vibration on the hand; detecting the vibration of the obturator includes detecting the vibration using a sensor coupled to the medical device; the sensor being coupled to the obturator; detecting the vibration using a sensor includes transmitting a signal representative of the vibration to a signal processing device; the signal processing device is configured to distinguish between a type of tissue that the obturator is piercing through based on the signal; the signal processing device is configured to identify proximity of the obturator with a bone in the body based on the signal; the signal processing device is configured to identify a contact of the obturator with a bone in the body based on the signal; contact of the obturator with a bone is detected based on detecting a frequency of a vibration induced in the obturator due to the contact; the sensor transmits the signal to the signal processing device wirelessly; the signal processing device is configured to indicate information relating to a position of the obturator within the body based on the signal; the information is indicated using an audio or a visual signal; the vibration generating device is embedded in the medical device; and the vibration generating device is embedded in the handle of the medical device.

It is to be understood that both the foregoing general description and the following detailed description are exemplary and explanatory only, and are not restrictive of the invention as claimed.

### BRIEF DESCRIPTION OF THE DRAWINGS

FIG. 1 is a schematic illustrating an exemplary laparoscopic surgical process.

FIG. 2 illustrates an exemplary trocar used in the laparoscopic process of FIG. 1.

FIGS. 3A-3C illustrate exemplary tip designs of the trocar of FIG. 2.

FIG. 4 illustrates another embodiment of a trocar used in the laparoscopic process of FIG. 1.

### DETAILED DESCRIPTION

Exemplary embodiments of the present disclosure are illustrated in the accompanying drawings. Wherever possible, the same reference numbers will be used throughout the drawings to refer to the same or like parts.

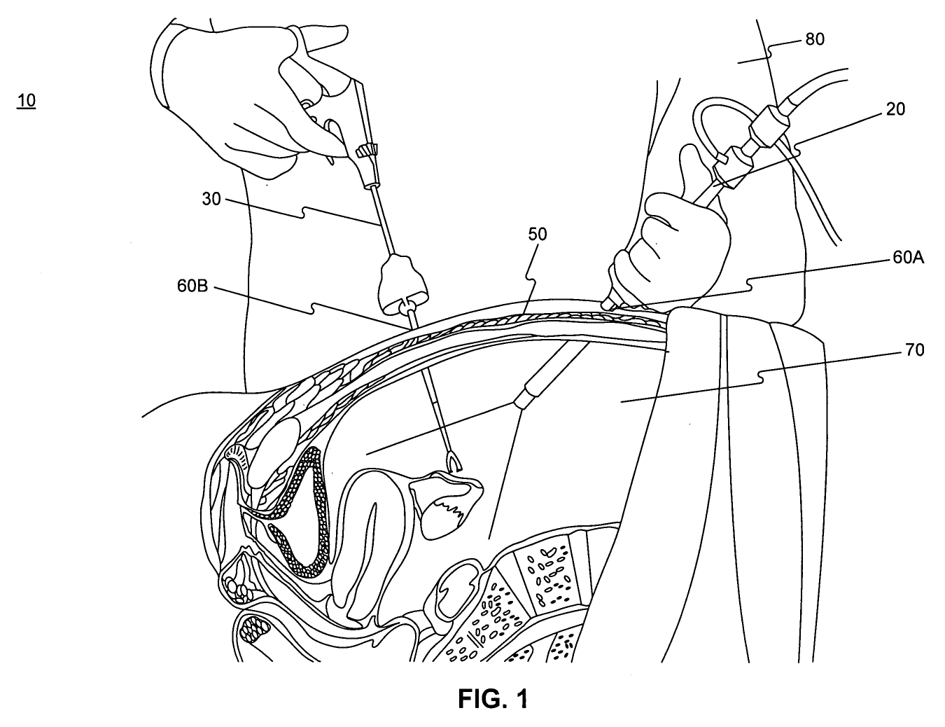

FIG. 1 depicts an exemplary laparoscopic surgical process 10 performed using surgical tools inserted into the abdominal cavity 70 through one or more punctures created on the abdominal wall 50. Non-limiting examples of the exemplary laparoscopic surgery may include cholecystectomies, gastrojejunostomies, stomach resections, polypectomies, vasectomies, tubal ligations, etc. It should be emphasized that the illustrated laparoscopic surgical process 10 of FIG. 1 is exemplary only, and the inventions of the current disclosure may be applied to any suitable surgical application known in the art.

To perform the laparoscopic surgical process 10 of FIG. 1, a laparoscope 20 is inserted into an abdominal cavity 70 through a primary puncture 60A on an abdominal wall 70. The primary puncture 60A may created by a controlled jab (as described earlier) of a trocar on the abdominal wall. The laparoscope 20 may be fitted with a camera, light and other capabilities that may enable a laparoscopist (surgeon 80) to view the procedure from outside the body. The laparoscope 20 may also include one or more lumens passing longitudinally therethrough. These lumens may enable surgical tools to be delivered to the abdominal cavity 70 to aid in the surgical procedure. In some surgical procedures, additional surgical tools (such as, for example, a grasper 30) may be delivered to the abdominal cavity 70 through a secondary puncture 60B created on the abdominal wall. Although one secondary puncture 60B is illustrated in FIG. 1, it is understood that different laparoscopic procedures may use a different number of secondary punctures. For instance, in some procedures, all the necessary surgical tools may be delivered to the abdominal cavity 70 through the laparoscope 20 and no secondary punctures may be needed, while in other procedures, more than one secondary puncture 60B may be needed. The secondary puncture 60B may also be created on the abdominal wall 50 using a trocar. The camera in laparoscope 20 may guide the surgeon 80 in the insertion of the trocar to create the secondary puncture 60B.

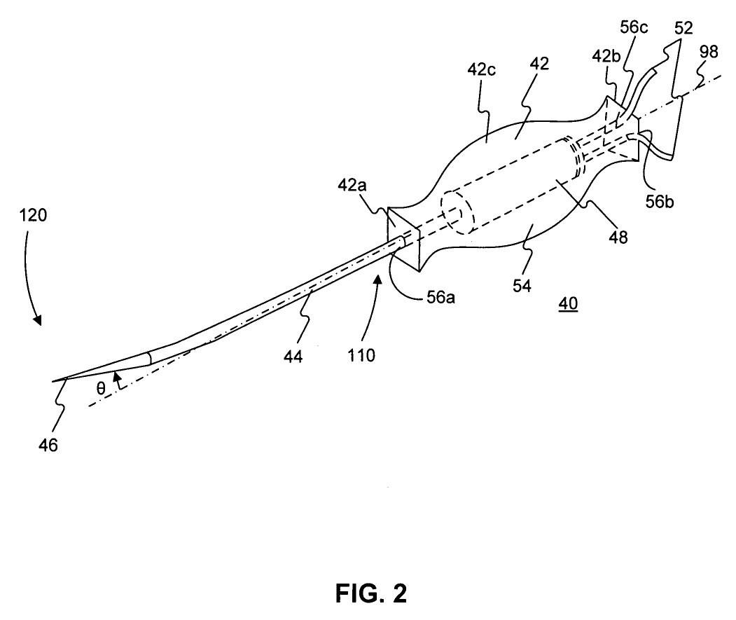

FIG. 2 illustrates an exemplary trocar 40 that may be used to create the punctures (primary puncture 60A and secondary puncture 60B). Trocar 40 may include a handle 42 with an elongate obturator 44 extending therefrom. Handle 42 may include a side surface 42c, positioned about a longitudinal axis 98, connecting a front wall 42a and a back wall 42b. Side surface 42c, front wall 42a, and back wall 42b may define a hollow cavity 54 within handle 42. In the embodiment of trocar 40 depicted in FIG. 2, side surface 42c is shown to curve both about the longitudinal axis 98 and along the longitudinal axis 98. This shape of side surface 42c may allow handle 42 to rest comfortably within a palm of a human hand. In general, it is contemplated that side surface 42c may have any general shape. For instance, in some embodiments, side surface 42c may have a substantially cylindrical shape.

Front wall 42a and back wall 42b may be opposing end faces of handle 42. In the embodiment of trocar 40 depicted in FIG. 2, front wall 42a and back wall 42b are both shown to have a substantially square shape. However, it is contemplated that, in general, front wall 42a and back wall 42b may have any general shape. For instance, in some embodiments, one or both of these surfaces may have a circular shape. Back wall 42b may include one or more openings that provide access to cavity 54, for instance a second opening 56b and a third opening 56c. Second opening 56b and third opening 56c may have any general cross-sectional shape and may be dimensioned to provide access to electrical cables wires into cavity 54. Front wall 42a may also include a first opening 56a that opens into cavity 54. Obturator 44 may extend into cavity 54 of handle 42 through first opening 56a. First opening 56a may have the same general shape as a cross-sectional shape of obturator 44. In some embodiments, an outer surface of obturator 44 may contact a mating surface of first opening 56a, while in other embodiments, sealing materials such as a bushing (such as a vibration damping bushing), may separate the mating surfaces of obturator 44 and first opening 56a.

Obturator 44 may include an elongate shaft extending from a proximal end 110 to a distal end 120 along longitudinal axis 98. At the proximal end 110, obturator 44 may extend into cavity 54 through first opening 56a. At the distal end 120, obturator 44 may terminate at a tip 46. Obturator 44 may have a generally circular cross-section, and may have different diameters for different applications. In general, obturator 44 may have any diameter found in obturators known in the art. The length of obturator 44 may also be similar to obturators known in the art. In some embodiments, obturator 44 may extend parallel to longitudinal axis 98, while in other embodiments, obturator 44 may include a curvature along its length. In the embodiment of trocar 40 shown in FIG. 2, obturator 44 is shown to extend substantially parallel to longitudinal axis 98, with a length of the obturator 44 at distal end 120 making an angle 0 with the longitudinal axis 98. Angle θ may vary from 0 degrees to about 90 degrees. In general, angle θ may depend upon the medical procedure (TVT, TNT, etc.) for which trocar 40 is used. For example, in an application where trocar 40 is used to deliver ends of a supporting tape (sling) to their respective locking positions, angle θ may vary with the shape of obturator 44 and the anchoring location. In embodiments where angle θ is 0 degrees, obturator 44 may extend from proximal end 110 to distal end 120, parallel to longitudinal axis 98.

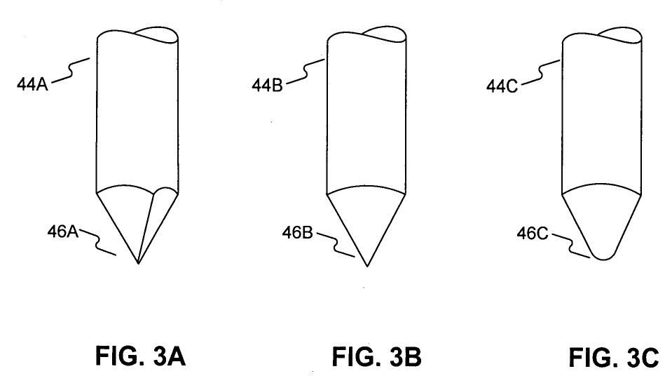

Tip 46 of the obturator may have any shape that is configured to puncture abdominal wall 50 (see FIG. 1). In general, tip 46 may be designed for sharp or blunt penetration. A few exemplary embodiments of tip 46 designed for sharp and blunt penetration are illustrated in FIGS. 3A-3C. Tip 46A of FIG. 3A has a pyramidal shape with multiple sides that terminate at a sharp point. Tip 46B of FIG. 3B has a conical shape terminating at a sharp point. FIG. 3C illustrates a conical tip 46C configured for blunt penetration. It should be emphasized that tips 46A-46C of FIGS. 3A-3C are exemplary only, and trocars of the current disclosure may include an obturator 44 having any type and shape of tip known in the art. Although not illustrated in FIG. 2 and FIGS. 3A-3C, in some embodiments, trocar 40 may also include a retractable shield that covers tip 46 before and after insertion. This shield may offer some protection to abdominal and pelvic organs from inadvertent puncture.

The proximal end 110 of obturator 44 in cavity 54 may be coupled to a vibrator 48. Vibrator 48 may include one or more devices that may be configured to impart mechanical vibration to obturator 44. These mechanical vibrations may include vibrations in the transverse and/or the longitudinal direction. In some embodiments, vibrator 48 may include an electric motor, a solenoid, and/or other electrical devices. Power and signals to and from the vibrator 48 may be provided by cables 52 that extend from handle 42 through second and third opening 56b, 56c in the back wall 42b. The vibrator 48, when activated by an electric current, would produce an alternative electric field. This alternative electric field may directly, or through some transmission parts (such as an offset wheel), impact longitudinal or transverse vibrations on the obturator 44. In general, the frequency of these vibrations may vary from a few Hertz to tens of kilohertz. The amplitude and frequency of the vibrations may be fixed or variable. In some embodiments of trocar 40, the frequency and/or amplitude of the vibration may be selected by the user depending upon the application. These vibratory characteristics may be selected, for example, by controlling the excitation current input to the vibrator 48. In some embodiments, handle 42 may include a knob or other selection device that enables the user to select the amplitude and/or frequency of vibration. In some embodiments, the amplitude and frequency of vibration may have a fixed value. This fixed value may depend upon the characteristics of vibrator 48.

The mechanical vibrations imparted to obturator 44 at the proximal end 110 may travel back and forth along the length of the obturator 44, and into handle 42. The vibration transmitted to handle 42 may be felt in the palm of surgeon 80 operating trocar 40. If the surgeon 80 holds trocar 40 in air, the vibration felt by the surgeon 80 may be a function of the amplitude and frequency of vibration of vibrator 48. Design parameters of trocar 40 (such as, size, material, damping in the system, etc.) may affect the mode of vibration. For example, if vibrator 48 is excited with an alternating field of 250 Hz at 10 W, the vibration felt by the surgeon 80 would depend upon the size and type of trocar 40. The vibration felt by the surgeon 80 will also depend upon the materials and construction of trocar 40. For instance, in a trocar with a 3 mm diameter and 200 mm long steel obturator 44, the vibration felt by surgeon 80 would be different if handle 42 were constructed out of a soft or a hard material. Likewise, presence of a soft bushing between obturator 44 and first opening 56a may also change the vibration felt by surgeon 80. Although the magnitude and/or frequency of the vibration felt by the surgeon may be amplified or dampened depending upon the construction of trocar 40, the vibration felt by surgeon 80 would not change over time, when trocar 40 is held in air.

As obturator 44 of trocar 40 penetrates abdominal wall 50 (to create a puncture), the tissue of abdominal wall 50 may absorb some of the energy, thereby reducing the amplitude of vibration. Although the amplitude changes as the obturator 44 penetrates tissue, the frequency of vibration may remain relatively unchanged. The surgeon 80 holding trocar 40 would feel the reduced amplitude of vibration. Depending upon the type of tissue and depth of obturator 44 penetration, the reduction in amplitude may be different. For instance, when obturator 44 penetrates soft tissue, the reduction in amplitude may be lower than when obturator 44 penetrates dense tissue or muscle layers. Additionally, the reduction in amplitude may also increase with increased depth of penetration. The surgeon 80 may get an indication of the location of tip 46 of obturator 44 by detecting variations in the amplitude of vibration felt in his/her hand.

When tip 46 of obturator 44 touches a bone, a second vibratory signal (or a shock wave) having a different frequency may be generated. This second vibratory signal may be the result of the impact of obturator 44 on bone. This second vibratory signal may also travel along the length of obturator 44 and be felt by surgeon 80. The different frequency and amplitude of the second vibratory signal, from the vibration generated by the vibrator 48, may enable the surgeon 80 to distinguish between the two vibratory signals. Using these vibratory signals as a guide, the surgeon 80 may guide trocar 40 through a desired path to create a puncture.

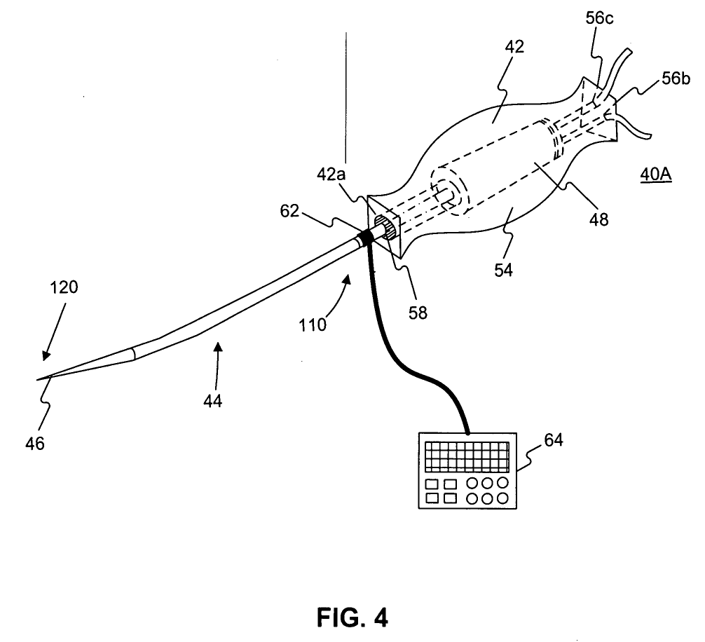

In some embodiments, a sensor 62 may be provided to detect the vibratory response of obturator 44. FIG. 4 illustrates an embodiment of trocar 40A with a sensor 62 coupled to obturator 44. Although FIG. 4 illustrates sensor 62 coupled to the proximal end 110 of obturator 44, sensor 62 may be coupled at any location of trocar 40A as long as the vibrations in obturator 44 can be detected by the sensor 62. Sensor 62 may be any type of device (for example, a pressure sensor, strain gage, accelerometer, etc.) that is known in the art. In some embodiments of a trocar 40A with sensor 62, vibration of the obturator 44 may be transmitted to the hand of surgeon 80, and be detected by the sensor 62. In these embodiments, the sensor 62 may serve as a back-up mechanism to detect vibratory signals that may be too weak to be detected by the surgeon 80. In other embodiments of trocar 40 with sensor 62, the handle 42 may be insulated from the obturator 44, such that the vibrations transmitted to the handle 42 may be minimized. In these embodiments, a layer of vibro-insulation or bushing 58 may be provided between the mating surfaces of obturator 44 and first opening 56a (or handle 42).

Sensor 62 may detect the vibrations of the obturator 44 and transmit them to a signal processing device 64. The signal processing device 64 may be embedded in trocar 40A or may be a separate unit. In embodiments where the signal processing device 64 is a separate unit, sensor 62 may convert the detected vibrations and transmit them wirelessly, or through a wired connection, to the signal processing device 64. In embodiments where the signal processing device 64 is embedded in the trocar 40A, the signal processing device 64 may be embedded anywhere in trocar 40A. In some embodiments the signal processing device 64 may also be located in the hollow cavity 54 of handle 42.

The signal processing device 64 may be configured to differentiate between different layers of body tissue (tissue, fat, tendons, muscle, bone etc.) that the obturator 44 is penetrating through, or is adjacent to. As an illustrative example, one method that may be used by the signal processing device 64 to differentiate between different layers of body tissue is described below. The obturator 44 may be excited by a harmonic vibration having an equation y(t)=Aₒ cos(ωt+φₒ), where y(t) is the displacement at time t, Aₒ is the amplitude of oscillation, ω is the frequency, t is the elapsed time, and φₒ is the phase of oscillation. The displacement at any point of the obturator 44, in response to this excitation, may be given by the equation x(t)=A cos(ωt+φ). While the frequency component (ωt) may remain a constant irrespective of the type of tissue that the obturator 44 is penetrating through, the amplitude and the phase delay components (A, φ) may be a function of the density of this layer of tissue. The higher the density of the tissue that obturator 44 is penetrating through, the lower would be the amplitude and higher would be the phase delay at any point on the obturator 44.

The signal processing device 64 may extract the difference in amplitude and/or phase delay from the sensor 62 signal. The signal processing device 64 may also include calibration data that quantifies the amplitude and phase delay for different types of tissue. Based on the sensor signal and the calibration data, signal processing device 64 may identify the location of the obturator tip 46 in the abdominal wall 50. If the tip 46 hits a bone, the second vibratory signal generated due to the contact may be captured by sensor 62. The second vibratory signal may be composed of frequency components that may be distinguishable from the vibration induced by vibrator 48. These different frequency components may be detected by the signal processing device 64. In some embodiments, the calibration data may also include data that indicates the proximity of the tip 46 to a bone. In these embodiments, signal processing device 64 may also indicate the proximity of the obturator 44 to a bone. It should be emphasized that the illustrated method of differentiating between different body tissue layers (and identifying the location of the obturator 44) by the signal conditioner is exemplary only. Any known method may be used identify the location of the obturator 44 in the body from the vibration signals detected by the signal processing device 64.

The signal processing device 64 may also be configured to indicate to surgeon 80 information relating to the location of the obturator 44 in the body. This information may be conveyed to surgeon 80 by any known means. In some embodiments, this information may be conveyed to surgeon 80 using a visual indicator, an audible signal, or a tactile signal. For instance, sound signals may indicate when the obturator 44 pierces through different layers of the abdominal wall. The pitch or intensity of the sound signal may change when the tip 46 approaches and/or contacts a bone. In some embodiments, a visual indicator (such as, lights, etc.) may indicate the location of the obturator 44. In some embodiments, the location of the obturator 44 may be displayed on a monitor overlaid on an image of the abdominal wall 50 to indicate the layer of tissue that the obturator 44 is piercing through. It is also contemplated that a combination of auditory, visual, and tactile signals may be used to relate information from the signal processing device 64 to surgeon 80.

Using a trocar 40 with vibratory feedback indicating the tissue layer that the trocar is piercing through, may serve to guide the surgeon 80 during insertion of trocar 40 during laparoscopic surgery. The feedback from the trocar 40 may help reduce the likelihood of injury due to blind insertion of trocar 40 during primary insertion. The sensor 62 combined with the signal processing device 64 may reduce the need for surgeon 80 to be sensitive to the variations in vibrations (felt in his/her hand holding the trocar) to guide the trocar 40A, while creating a puncture. Additionally, the vibrating obturator 44 may improve the ability of the trocar 40 to pierce through tissue, thereby reducing the force needed to pierce tissue and advance the obturator 44 through the tissue.

An exemplary method of using a trocar 40 of the current disclosure to create a puncture on a body surface will now be explained. With the handle 42 of trocar 40 firmly grasped by a hand of surgeon 80, the tip 46 of trocar 40 may be positioned proximate the body surface. The power to the vibrator 48 may be switched on to start the vibration of the obturator 44. The surgeon may now be able to feel the vibration on his hand holding the trocar 40. In embodiments of trocar 40 fitted with sensor 62 to detect the vibratory response, these vibrations may alternatively or additionally be detected by the signal processing device 64 as a reference signal. The trocar 40 may be moved towards the body surface so that the tip 46 of the obturator 40 pierces through the body surface. As the trocar 40 pierces through the body surface, the obturator 44 travels through different layers of body tissue. As the obturator 44 proceeds through different layers of body tissue, the surgeon 80 will feel variations in the amplitude of vibration through the handle 42 of the trocar 40. In embodiments of trocar 40 with sensor 62, the signal processing device 64 may indicate the layer of tissue that the obturator 44 is piercing through. Using this feedback from the trocar 40 as a guide, the surgeon 80 may maneuver the trocar 40 into the body while avoiding internals organs and parts of the body that he/she wishes to avoid. If the obturator 44 contacts a bone, the impact of the obturator 44 on the bone may induce a second vibratory signal on the obturator 44. This second vibratory signal may be a shock wave or a vibration having a different frequency than the reference vibration, and may be detected by the surgeon and/or the signal processing device 64. When the surgeon 80 detects that the obturator 44 has contacted a bone, he may reposition the trocar 40 within the body to avoid the bone.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

We claim:

1. A medical device, comprising:
a handle configured to be grasped by a human hand;
an elongate obturator coupled to the handle, the obturator including a proximal end proximate the handle and a distal end extending away from the handle, the distal end of the obturator including a tip configured to pierce a body; and
a vibration generating device coupled to the obturator, the vibration generating device being configured to induce a vibration having a frequency and an amplitude on the obturator.

2. The medical device of claim 1, wherein the vibration generating device is embedded in the medical device.

3. The medical device of claim 1, wherein the obturator is configured to change the amplitude of vibration of the obturator as the obturator pierces the body.

4. The medical device of claim 3, wherein the medical device is further configured to transmit the vibration to the handle.

5. The medical device of claim 3, wherein the obturator is configured to vary the amplitude of vibration as a function of a density of a layer of tissue that the obturator pierces through.

6. The medical device of claim 1, wherein the proximal end of the obturator extends into the handle.

7. The medical device of claim 1, wherein the vibration generating device is physically coupled to the obturator.

8. The medical device of claim 1, wherein the vibration generating device includes at least one of an electric motor and a solenoid.

9. The medical device of claim 1, further including a sensor coupled to the obturator, wherein the sensor is configured to detect a vibration of the obturator.

10. The medical device of claim 9, wherein the sensor includes one of a pressure sensor and an accelerometer.

11. The medical device of claim 9, wherein the sensor transmits signals indicative of the detected vibrations to a signal processing device.

12. The medical device of claim 11, wherein the signal processing device is embedded in the medical device.

13. The medical device of claim 12, wherein the signal processing device is embedded in the handle.

14. The medical device of claim 11, wherein the signal processing device is a standalone device separate from the medical device.

15. The medical device of claim 11, wherein the sensor wirelessly transmits the signals to the signal processing device.

16. The medical device of claim 11, wherein the signal processing device is configured to indicate information related to the position of the obturator within the body based on the signal.

17. The medical device of claim 16, wherein the information includes the type of tissue that the obturator pierces through.

18. The medical device of claim 16, wherein the information is indicated using a visual or an audio signal.

19. The medical device of claim 11, wherein the signal processing device is configured to indicate the proximity of the obturator to a bone in the body as the obturator pierces the body.

20. The medical device of claim 11, wherein the signal processing device is configured to indicate a contact of the obturator with a bone in the body.

21. The medical device of claim 1, further including a vibration isolation bushing between the obturator and the handle.

22. A method of using a medical device to create a puncture in a body comprising:
grasping a handle of the medical device with a hand, the handle being coupled to an elongate obturator;
activating a vibration generating device coupled to the obturator, the vibration generating device inducing a vibration having a frequency and an amplitude on the obturator;
piercing the body with the obturator;
detecting a vibration of the obturator during the piercing; and
controlling the piercing of the body based on the detected vibration of the obturator.

23. The method of claim 22, wherein controlling the piercing further includes detecting a variation of the amplitude of the vibration as the obturator pierces into the body.

24. The method of claim 22, wherein controlling the piercing further includes detecting a frequency of vibration induced in the obturator.

25. The method of claim 22, wherein controlling the piercing further includes identifying the type of tissue the obturator is piercing through based on the vibration of the obturator.

26. The method of claim 22, wherein controlling the piercing further includes detecting contact of the obturator with a bone in the body based on the vibration of the obturator.

27. The method of claim 22, wherein detecting the vibration of the obturator includes detecting the vibration on the hand.

28. The method of claim 22, wherein detecting the vibration of the obturator includes detecting the vibration using a sensor coupled to the medical device.

29. The method of claim 28, wherein the sensor is coupled to the obturator.

30. The method of claim 28, wherein detecting the vibration using a sensor further includes transmitting a signal representative of the vibration to a signal processing device.

31. The method of claim 30, wherein the signal processing device is configured to distinguish between a type of tissue that the obturator is piercing through based on the signal.

32. The method of claim 30, wherein the signal processing device is configured to identify proximity of the obturator with a bone in the body based on the signal.

33. The method of claim 30, wherein the signal processing device is configured to identify a contact of the obturator with a bone in the body based on the signal.

34. The method of claim 33, wherein contact of the obturator with a bone is detected based on detecting a frequency of a vibration induced in the obturator due to the contact.

35. The method of claim 30, wherein the sensor transmits the signal to the signal processing device wirelessly.

36. The method of claim 30, wherein the signal processing device is configured to indicate information relating to a position of the obturator within the body based on the signal.

37. The method of claim 36, wherein the information is indicated using an audio or a visual signal.

38. The method of claim 22, wherein the vibration generating device is embedded in the medical device.

39. The method of claim 38, wherein the vibration generating device is embedded in the handle of the medical device.
